(19) United States
(12) Patent Application Publication
(10) Pub. No.: US 2011/0056126 A1
(43) Pub. Date: Mar. 10, 2011

(54) PROCESS FOR PRODUCING HIGH VALUE PRODUCTS FROM BIOMASS

(76) Inventors: J. T. Harvey, Lakewood, CO (US); Timothy P. Spilchen, Green Bay, WI (US); Andrew W. Flemming, Littleton, CO (US); Lisa B. Andersen, Lakewood, CO (US); John H. Evans, IV, Superior, CO (US); Christine A. Singer, Lakewood, CO (US)

(21) Appl. No.: 12/856,576

(22) Filed: Aug. 13, 2010

Related U.S. Application Data
(60) Provisional application No. 61/233,824, filed on Aug. 13, 2009.

(51) Int. Cl. C10L 5/44; C12P 7/06

(52) U.S. Cl. 44/606; 435/161

(57) ABSTRACT

A process for converting biomass into high value energy products is provided comprising the steps of: debarking, chipping, and screening wood; separating a plurality of hemicellulose from a plurality of cellulose and lignin; hydrolyzing the plurality of hemicellulose into monosaccharides; fermenting the monosaccharides using immobilized Pachysolen tannophilus; removing the lignin from the plurality of cellulose and lignin; and making a paper pulp from the plurality of cellulose.

This application claims the benefit of U.S. Provisional Application No. 61/233,824, filed Aug. 13, 2009, which is hereby incorporated by reference.

### FIELD

The present patent document relates to an apparatus and process for producing high value products from biomass.

### BACKGROUND

Paper pulping is an old technology that has been around since the 1800's. The process of mechanically creating paper from wood was developed in Germany in the 1840's and chemical processing quickly followed. U.S. Pat. No. 70,485 was issued to Tilghman in 1867 for the process of using sulphurous acid to make paper pulp from vegetable substances.

The sulfate, or Kraft process, which is still the most popular method of pulping today, was developed by Carl F. Dahl in 1879. In addition to the Kraft process, numerous other chemical pulping processes exist today, including the sulfite process. The Kraft process is the most popular because it is thought to produce a stronger pulp than other pulping processes. In addition, the Kraft process also works on a wide range of wood and non-wood sources.

Paper pulp, and ultimately paper, is made primarily from the cellulose found in wood and other biomass. The chemical pulping processes deconstructs the wood, and/or other biomass, into pulp containing mainly pure cellulose fibers and varying concentrations of lignin, depending on the quality of the paper desired.

Wood, like other biomass, is made up primarily of cellulose, hemicellulose, and lignin bound together as a polymer network. The pulping process breaks down these bonds to allow the cellulose to be separated out from the lignin and hemicellulose, and made into pulp. Pulping processes attempt to separate the hemicellulose and lignin from the cellulose with as little degradation to the cellulose fibers as possible.

The chemical processing of wood or other biomass into pulp starts with a material preparation step. The wood starts by being debarked. Typically, only the heartwood and sapwood are used for making pulp. The structure of bark does not lend itself to pulping and is therefore removed and used as fuel to provide steam for use in the pulp mill. In most pulping processes, the wood is chipped and screened to provide uniform sized chips.

The wood chips are fed into vessels called digesters which may operate in batch or continuous mode. In the Kraft process, a mixture including sodium hydroxide and sodium sulfide is added to the wood chips. The digesters heat the mixture and wood from 130° C. to 180° C. In this temperature range, delignification may take up to several hours. Under these conditions, lignin and some hemicellulose degrade to give fragments that are soluble in the strongly basic liquid. The post process liquid mixture, known as black liquor (so called because of its color), contains lignin fragments, carbohydrates from the breakdown of hemicellulose, sodium carbonate, sodium sulfate and other inorganic salts.

By the time the wood has been debarked, and the lignin and hemicellulose has been removed, only about 30% of the original source material is turned into usable paper pulp. The black liquor containing the hemicellulose and lignin is combusted providing a recycle stream of reagents (called white liquor) and energy for the process. The hemicellulose component is heavily degraded in the Kraft pulping process into a less useful energy product. Therefore, even though the hemicellulose is contained in the black liquor, most of the black liquor's calorific energy is derived from the lignin. The bark and other tree parts are either used as fuel or left behind in the forest. As a result, the energy contained in the paper pulping byproducts is not typically efficiently utilized in current paper pulping processes.

Paper pulping is not the only current commercial process that leaves a significant amount of hemicellulose as a byproduct. For example, in the creation of sugar from sugarcane, bagasse is the fibrous residue remaining after the sugarcane is crushed to extract its juice. The bagasse is further used for a number of different purposes including being burned as fuel for the sugar mill, as a renewable resource in the manufacture of pulp and paper products and as building materials. Similar to the byproducts in other commercial processes like paper pulping, sugarcane bagasse is rich in polysaccharides.

It has long been known that the sugars found in biomass, for example sugarcane bagasse and wood, may be converted into ethanol and other fuel products. In addition, paper pulping processes and biomass ethanol conversion processes both begin with the same basic step of breaking down the bonds between the lignin, cellulose and hemicellulose. A significant difference between the processes, however, is the fact that the component of the wood or biomass that may be easily converted into ethanol or other fuels is cellulose, the very product used to make the paper pulp in a pulping process. As already discussed, cellulose constitutes the portion of the wood or biomass extracted and used in paper. Because cellulose is the primary component of both ethanol production and paper pulping, and cellulose requires the most severe treatment to be liberated, the two processes have been mutually exclusive.

Unlike cellulose, which is a homogeneous polysaccharide of glucose molecules, hemicellulose is a heterogeneous polymer containing a mixture of hexose and pentose sugars. Hemicellulose generally contains the hexoses mannose, glucose, and galactose and the pentoses xylose and arabinose. Notably, mannose is the most abundant molecule in hemicellulose polymers derived from softwoods; the second most abundant sugar molecule in softwood hemicellulose being xylose. More generally, the hemicellulose of hardwoods and herbaceous crops and non-woody agricultural waste, such as sugarcane bagasse, are primarily enriched in the pentose xylose and the hexose glucose, with minor amounts of arabinose, mannose, and galactose.

The pentoses xylose and arabinose have traditionally been more difficult to convert to fuel products than glucose, mannose, or galactose (six carbon sugars). For a long time it was thought that yeast strains could not anaerobically ferment pentoses into alcohol. However, U.S. Pat. No. 4,359,534 to Kurtzman et al. discloses the use of Pachysolen tannophilus to ferment pentose. Similarly, U.S. Pat. No. 7,344,876 to Levine discloses a pure culture of Kluyveromyces marxianus capable of proliferation on pentose as the sole carbon source.

While the patents to Kurtzman and Levine disclose the use of yeasts for fermentation of pentoses into ethanol, commercial applications have been limited because of poor efficiencies. Yeasts and other microbes that can ferment xylose and other pentoses in a controlled or artificial medium, generally perform poorly in acid hydrolysates. Challenges presented by biomass hydrolysate include an acidic pH and a high concentration of toxic compounds, including acetic acid, phenolic compounds, 5-hydroxymethylfurfural (HMF) and furfural, and other inhibitory molecules produced during hemicellulose hydrolysis.

Another challenge of microbial conversion of sugars to ethanol is that many microbes such as yeast and bacteria are selective in the sugars they metabolize. For example, microbes that may convert the hexose glucose to ethanol may fail to convert other hexoses, such as mannose and galactose. Similarly, microbes that convert the pentose xylose to ethanol do not convert the pentose arabinose. Furthermore, the rates of conversion to fuel of mannose, galactose, glucose, and other hexoses to fuel differ among different microbial species.

Despite the inefficiencies, the possibility of removing hemicellulose from the paper pulping process and converting it to ethanol was hypothesized by the Georgia Institute of Technology in W. J. Fredrick et al., Co-production of ethanol and cellulose fiber from Southern Pine: A technical and economic assessment, 32 Biomass and Bioenergy 1293-1302 (2008). However, Fredrick notes that the 85% conversion rate of pentoses to ethanol “is an optimistic estimate that assumes that on-going research will make it possible.” The study concludes that ethanol production from loblolly pine may not be competitive with ethanol from other lignocellulosic sources when it is co-produced with cellulose fiber.

In addition to the lack of an efficient process that can ferment pentoses, other problems prevent ethanol conversion and paper pulping processes from being combined. For example, yeasts that ferment pentoses into ethanol are, relatively ethanol intolerant, ferment pentoses at a lower metabolic rate than hexoses, may not ferment all hexoses found in woody and herbaceous hydrolysates, may produce xylitol as a product of xylose metabolism, and may have strict nutrient and oxygen requirements. These qualities make pentose-fermenting microbes such as yeast and bacteria difficult to work with. Furthermore, biomass hydrolysate, for example the hydrolysate produced during the pulping process or acid hydrolysis of sugarcane bagasse, is typically toxic to microbes that are known to ferment pentoses. In addition, for processes that create an end product that is dependant on maintaining the integrity of the fibrous cellulosic material like paper pulping, care must be taken to remove the hemicellulose without substantial removal or degradation of cellulose. However, cellulose degradation is much less of a concern when the cellulose of the biomass feedstock is not intended to be used in a paper product.

### SUMMARY

In view of the foregoing, an object according to one aspect of the present patent document is to provide an improved process for converting the byproducts of paper pulping and other biomass processes into one or more usable products of higher value. It is also a separate object of present patent document to provide a process for converting a byproduct of sugar production, namely sugarcane bagasse, into one or more useable products or higher value.

Preferably the processes described herein address, or at least ameliorate, one or more of the problems described above. To this end, in one aspect, a process for converting wood into biofuel and paper pulp is provided comprising the steps of: producing a liquid hydrolysate comprising hemicellulose hydrolysate and a biomass residue from debarked wood chips; separating liquid hydrolysate from the biomass residue; fermenting monosaccharides in the separated liquid hydrolysate using an immobilized fermentative microbe into biofuel; and removing lignin from the biomass residue to form a paper pulp.

The fermented biofuel may, for example, comprise an alcohol like ethanol or butanol. In another embodiment, the producing step comprises the step of cooking the wood chips in a pressure reactor. Pressure reactors can liberate the hemicellulose from the cellulose and lignin without substantial degradation to the cellulose.

In yet another embodiment, the separating step comprises pressing the biomass residue or wood chips to express a portion of the liquid hydrolysate from the biomass residue or cooked wood chips. At the same time a portion of the liquid hydrolysate is being expressed, the pressing may form the biomass residue or cooked wood chips into a high energy biofuel or paper mill feedstock alternative.

In another embodiment, the immobilized fermentative microbe is Pachysolen tannophilus and the Pachysolen tannophilus is immobilized, for example, in calcium alginate. Immobilization increases the effectiveness of fermentative microbes, such as Pachysolen, and reduces the microbe's sensitivities to inhibitors found in the liquid hydrolysate. The immobilization may be performed using numerous methods including, but not limited to, forming the calcium alginate into beads in the range of 0.1 mm to 5 mm in diameter, more preferably 2 mm to 3 mm in diameter, and even more preferably about 3 mm in diameter.

In another embodiment, more than 80% of the monosaccharides in the separated liquid hydrolysate are converted to ethanol.

In yet another embodiment, the biomass residue is not processed into paper but instead is formed into a solid high energy density product. Preferably, the solid high energy density product is formed by pressing. Because pressing may also be used to perform the separating step, forming the solid high energy density product and separating the liquid hydrolysate may occur in the same pressing step or in separate steps. In addition, other types of biomass fiber sources may be used other than wood for the same process. For example, sugarcane bagasse is a biomass fiber source that may be used to produce paper pulp or a solid high energy density product.

In another embodiment, a high energy density biofuel comprises a compacted biomass residue including cellulose and lignin which is substantially free of hemicellulose. Preferably the high energy density biofuel has an energy density greater than 7,000 Btu/lb. However, the high energy density biofuel may have an energy density between 4000 Btu/lb and 10,000 Btu/lb depending on the water content. To this end, the compacted biomass preferably has a water content of less than about 45%, and more preferably less than about 25%, but may have a higher water content provided the energy density of the biofuel remains sufficiently high.

In yet another embodiment, the compacted biomass comprises less than 10% hemicellulose by weight.

In another aspect of the present patent document, a process for converting a biomass fiber source into biofuel and a high-value product is disclosed. The process comprises producing a liquid hydrolysate comprising hemicellulose hydrolysate and a biomass residue from the biomass fiber source; separating liquid hydrolysate from the biomass residue; fermenting monosaccharides in the separated liquid hydrolysate using at least one fermentative microbial species immobilized in an immobilization medium into a biofuel; and creating a high-value product from the biomass residue.

The fermented biofuel may, for example, comprise an alcohol like ethanol or butanol. The high-value product may be paper, a paper mill feedstock alternative, or a high-energy density product. The biomass fiber source may be any biomass that provides a suitable source of cellulose for paper products, including, for example, wood and bagasse.

In one embodiment, the at least one fermentative microbial species includes at least two different microbial species with complimentary fermentation characteristics. The complimentary fermentation characteristics may be any characteristic, for example each microbe may be better at fermenting a different monosaccharide or each species may have a different metabolic rate. Where more than one microbial species is present, the species may comprise both a yeast species and a bacteria species.

When more than one fermentative microbial species is employed, each of the microbial species may be immobilized in the same medium, or alternatively, they may be immobilized in a separate medium. For example, each microbial species could be immobilized in the same or separate calcium alginate beads. If each microbial species is immobilized in separate beads, beads with each of the immobilized species may then be combined or added to the same fermentation vessel. Alternatively, the beads may be kept in separate fermentation vessels that are arranged in series to one another so that the liquid hydrolysate may be passed through each vessel in series to subject the hydrolysate to fermentation by each microbial species.

In other embodiments, the process may also include an additional step of conditioning the liquid hydrolysate after the hydrolysate is separated from the biomass residue to reduce the level of inhibitory secondary products contained in the hydrolysate. As part of the conditioning step, secondary products having a high value may be removed from the hydrolysate and then recovered. High value secondary products may include, but are not limited to, sulfuric acid, acetic or other organic acids, anti oxidants (including, for example, phenolic compounds, polyphenolic compounds liberated from the partial hydrolysis of lignin), nutraceutical products, cosmeceutical products, pharmaceutical products, furans, furfural, and 5-hydroxymethylfurfural.

Suitable methods for removing high value secondary products of interest for subsequent recovery include filtration, adsorption, and/or ion exchange. Other techniques, however, may also be used to the extent they permit removal of high value secondary products of interest and their subsequent recovery.

In yet another aspect of the present patent document, a process for converting sugarcane bagasse into biofuel is disclosed. The process comprises the steps of: producing a liquid hydrolysate comprising hemicellulose hydrolysate and a biomass residue from sugarcane bagasse; separating liquid hydrolysate from the biomass residue; fermenting monosaccharides in the separated liquid hydrolysate using an immobilized fermentative microbe to biofuel; and reducing the moisture content of the biomass residue to produce a high-energy density biofuel.

The process may also include an additional step of conditioning the liquid hydrolysate after the hydrolysate is separated from the biomass residue to reduce the level of inhibitory secondary products contained in the hydrolysate. As part of the conditioning step, secondary products having a high value may be removed from the hydrolysate and then recovered.

As described more fully below, the processes described herein may be used to efficiently convert a biomass fiber source into biofuel and another high-value product. For example, in one particular implementation paper pulping byproducts are converted into biofuel and another high-value product. In another example, sugarcane bagasse is converted into biofuel and another high-value product. Further aspects, objects, desirable features, and advantages of the methods disclosed herein will be better understood from the detailed description and drawings that follow in which various embodiments are illustrated by way of example. It is to be expressly understood, however, that the drawings are for the purpose of illustration only and are not intended as a definition of the limits of the claimed invention.

### BRIEF DESCRIPTION OF THE DRAWINGS

FIG. 1 illustrates a process for producing biofuel and/or ethanol and paper pulp from wood.

FIG. 2 illustrates another embodiment of a process for producing biofuel and/or ethanol and a high value produce from a biomass fiber source.

FIG. 3 illustrates process for recycling a calcium alginate immobilization medium.

FIG. 4 illustrates another process for producing biofuel and/or ethanol and paper pulp from wood.

FIG. 5 illustrates a process for making a biofuel, such as alcohol, including, for example, ethanol, and solid biofuel from a lignocellulosic biomass.

FIG. 6 illustrates the energy density increase in biomass residue as the percentage of hemicellulose removal is increased for a given moisture content of 25%.

FIG. 7 illustrates the energy density increase in wood biomass residue as the percentage of hemicellulose removal is increased for a given moisture content of 40%.

FIG. 8 illustrates the change in energy density of the residue as the percentage of water is reduced.

FIG. 9 illustrates the change in energy density of softwood residue with 75% hemicellulose conversion as the percentage of water is reduced.

FIG. 10 illustrates the change in total available energy of the residue over a range of moisture contents at three different levels of hemicellulose conversion (65%, 75%, and 85%).

FIG. 11 illustrates an energy balance flow diagram for treating bagasse in accordance with one embodiment of the present patent document.

FIG. 12 illustrates an energy balance flow diagram for treating wood in accordance with one embodiment of the present patent document.

FIG. 13 is a graph illustrating ethanol yield for regenerated calcium alginate beads with immobilized fermentative microbes over a series of fermentations.

### DETAILED DESCRIPTION

In the following description of the preferred embodiments, reference is made to the accompanying drawings that form a part hereof, and in which is shown by way of illustration specific embodiments in which the invention may be practiced. It is to be understood that other embodiments may be utilized and structural changes may be made without departing from the scope of the present invention.

Consistent with its ordinary meaning as a renewable energy source, the term “biomass” is used herein to refer to living and recently dead biological material including carbohydrates, proteins and/or lipids that may be converted to fuel for industrial production. By way of non-limiting example, “biomass” refers to plant matter, including, but not limited to switchgrass, sugarcane bagasse, corn stover, corn cobs, alfalfa, Miscanthus, poplar, and aspen, biodegradable solid waste such as dead trees and branches, yard clippings, recycled paper, recycled cardboard, and wood chips, plant matter listed above or animal matter, and other biodegradable wastes.

In the pulp and paper industry, wood is typically pretreated in a pulping process where the hemicellulose and lignin are removed leaving a high value cellulose product for paper making. In current known processes, the hemicellulose is not utilized in the paper process except as a small energy contributor to the black liquor. The processes proposed in the present patent document utilize the hemicellulose of the wood feed and/or wood waste for the production of ethanol and other biofuels, including other alcohols like butanol. The additional process steps may be inserted into the existing pulp mill process in a number of different ways to minimize capital investment in new equipment. Preferably, the new process steps are inserted after log debarking and chipping but prior to chemical or mechanical pulping.

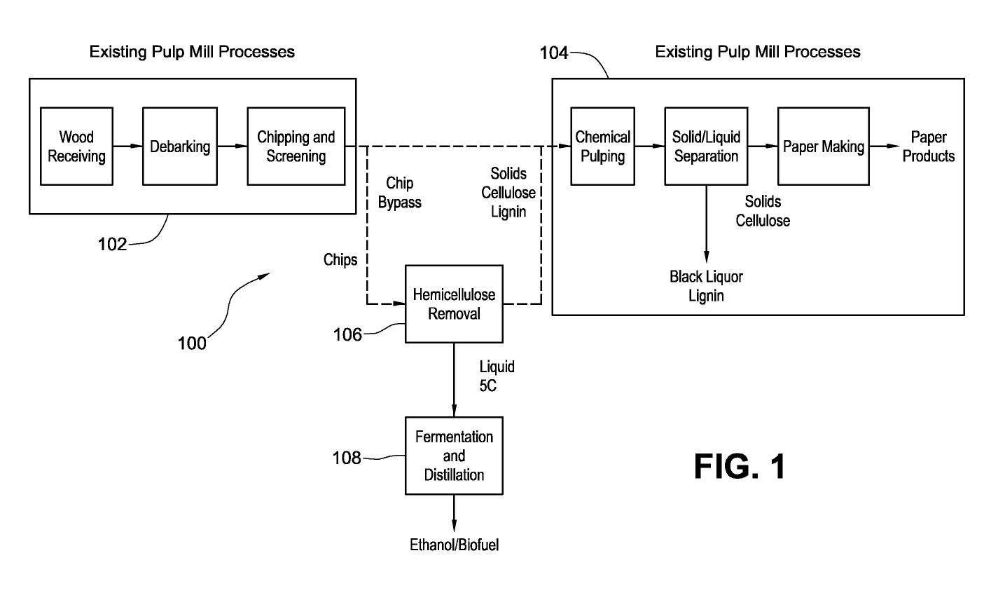

FIG. 1 illustrates a process for producing ethanol and paper pulp from wood. Process 100 includes the steps of a typical chemical paper pulping process 102 and 104. In wood conditioning step 102, the wood is received, debarked, chipped, and screened just as it normally would be in a chemical wood pulping plant. Most pulp mills and forest product plants have some form of wood handling systems. A complete wood lot may include a log handling system for unloading trees, a debarking system, and a wood chipping system. A stockpile and reclaim system are often also employed. For mills desiring increased pulp quality, chip sizing is important and therefore, most sites include a chip screening system. Some pulp mills utilize pre-chipped wood feed delivered by truck. These sites may also include specialized truck off-loading systems.

The wood conditioning steps shown in FIG. 1 are a common part of the wood conditioning step 102 of most paper pulping processes. However, although chipping and screening are a common part of the conditioning of most paper pulping processes, they are not a requirement of the conditioning process for the processes disclosed herein.

Furthermore, although the wood conditioning steps depicted in FIG. 1 are referring to wood, other types of biomass may be used, particularly other biomass fiber sources that have conventionally been used in the paper pulping industry. If non-wood sources of biomass are used different conditioning steps may be needed.

The processes described in the present patent document may also utilize more of the source tree than typically used during paper pulping. For example, the bark and other tree parts known as hog fuel or wood waste are typically not used in the process for making pulp, but may be used for biofuel and/or ethanol production in certain embodiments of the processes described herein.

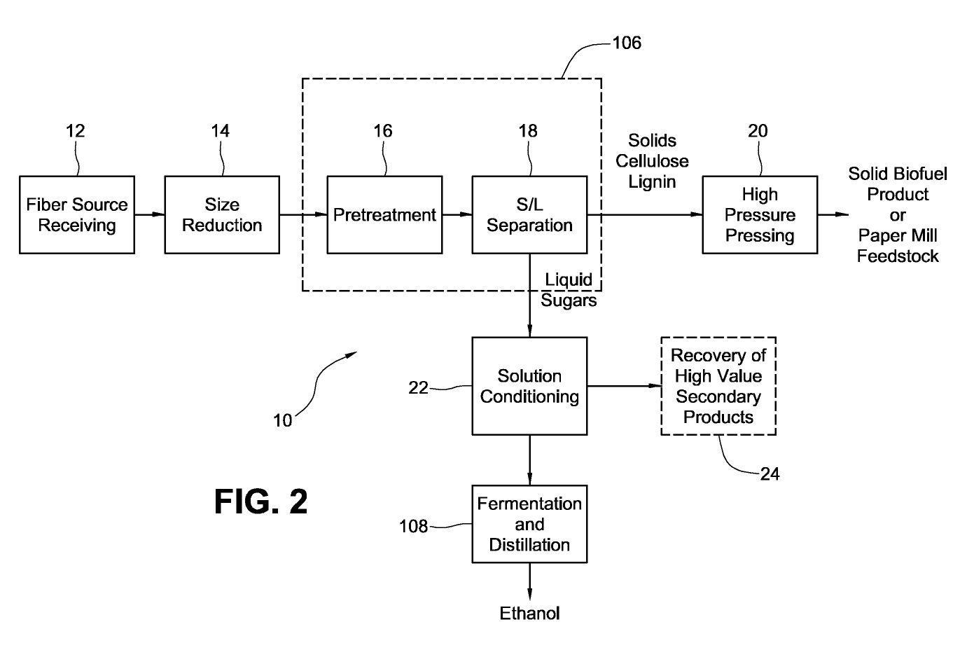

Once the wood or other biomass has been conditioned and is ready for paper pulping, some or all of the chips may be routed to undergo the additional process steps inserted into the overall paper process as shown in FIG. 1. The additional process steps include hemicellulose removal 106, which separates the hemicellulose from the biomass and solubilizes the pentoses and hexoses. The solubilized sugars may then be separated from the biomass residue in a liquid hydrolysate and fermented into ethanol or other biofuel products in step 108. As explained in more detail below in connection with FIG. 2, fermentation of the monosaccharides in the separated liquid hydrolysate is performed in step 108 using at least one fermentative microbial species immobilized in an immobilization medium into a biofuel. Any suitable solid/liquid separation technology may be used to perform the separation. The lignin and cellulose residue from step 106 continues on to be made into a high value product such as paper through a conventional paper pulping process 104 as illustrated in FIG. 1. In other embodiments, however, the lignin and cellulose residue may be made into a high-energy density fuel or a paper mill feedstock.

Another embodiment of a process according to the present patent document is illustrated in FIG. 2. The process 10 illustrated in FIG. 2 is a process for converting a biomass fiber source 12 into biofuel and a high-value product. The fermented biofuel may, for example, comprise an alcohol like ethanol or butanol. The high-value product may be paper, a paper mill feedstock, or a high-energy density product. The biomass may be any suitable lignocellulosic biomass. More preferably, the biomass comprises a biomass fiber source, such as wood or bagasse, that provides a suitable source of cellulose fibers for paper products. Thus, the process 10 illustrated in FIG. 2 is more generalized than the process 100 of FIG. 1 in terms of its depicted feedstock and its output. Because process 10 is more generalized, it may be inserted into or appended to a wider variety of existing commercial biomass processing plants, including, for example, sugarcane processing plants and paper pulp mills, such as the paper pulp mill process illustrated in FIG. 1.

The process 10 comprises the steps of: producing a liquid hydrolysate comprising hemicellulose hydrolysate and a biomass residue from the biomass fiber source in pretreatment step 16, separating liquid hydrolysate from the biomass residue in step 18, fermenting monosaccharides in the separated liquid hydrolysate using at least one fermentative microbial species immobilized in an immobilization medium into a biofuel in step 108, and creating a high-value product from the biomass residue in step 20. Hemicellulose removal step 106 of FIG. 1 comprises pretreatment steps 16 and solid/liquid separation step 18 in FIG. 2 as reflected by the dashed box around these two steps in FIG. 2.

In optional step 14, the biomass fiber source may be reduced in size as already explained with respect to the application of the process to the paper pulping industry (see FIG. 1). If the biomass fiber source is received in a size that is already appropriate for treatment in process 10, as those skilled in the art will appreciate from the description herein, further sizing will not be required.

Once the biomass is the appropriate size, it often needs to undergo some form of process to disrupt the polymer network of cellulose, hemicellulose, and lignin forming the biomass structure so the polysaccharides can be reduced to monosaccharides. This process is commonly referred to as “pretreatment” as shown in step 16 of FIG. 2. The pretreatment step 16 is designed to reduce the recalcitrance of the biomass to enzymatic or chemical saccharification of at least the hemicellulose contained therein. In some embodiments, however, the pretreatment may also reduce the recalcitrance of both the hemicellulose and cellulose in the biomass to enzymatic or chemical saccharification. Furthermore, in some embodiments the pretreatment may go further and also be responsible for saccharification of the hemicellulose and/or cellulose into their monosaccharide components. A liquid hemicellulose hydrolysate may be produced from wood or other biomass during pretreatment 16 using a number of methods including, for example, in a pressure reactor. Table 1 lists appropriate ranges for temperature, dwell time, and moisture content necessary for the separation and hydrolyzation of hemicellulose in a pressure reactor.

TABLE 1
Pressure Reactor Pretreatment Conditions

| Temperature* | 105-200° C. |
|---|---|
| Dwell time | 1 minute-24 hours |
| Moisture Content | 25-90% |

*Temperature dictates the pressure in a sealed vessel in a saturated steam environment

Reagents may be used to enhance the effectiveness of the pretreatment. Different biomass sources may respond better to the addition of different reagents. Reagents may include, but are not limited to: nitric acid, phosphoric acid, hydrochloric acid, sulphuric acid, sulphur dioxide, and sodium sulphite. Other reagents that reduce the recalcitrance of the biomass to hemicellulose removal may also be added.

In addition to performing the pretreatment step 16 in a pressure reactor, pretreatment step 16 may be performed using a number of other methods including acid prehydrolysis, steam cooking, alkaline processing, rotating augers, steam explosion, and ball milling. One advantage of a pressure reactor is that it not only liberates or extracts the hemicellulose but the pressure reactor can also facilitate the breakdown of the hemicellulose and solubilize the pentoses and hexoses at the same time to form a hemicellulose hydrolysate. This eliminates the need to add large amounts of enzymes.

Once hemicellulose is liberated from the biomass and the monosaccarides are solubilized, fermentation can begin. While fermentation may occur within the biomass residue, preferably the sugars are separated in step 18 via solid/liquid separation and/or washing them from the biomass residue and then fermented ex-situ in step 108. A preferred method of fermentaton of liquid hydrolysate comprising a hemicellulose hydrolysate is described in U.S. Provisonal Patent Application Ser. No. 61/233,821 and U.S. patent application Ser. No. 12/856,566, both of which are hereby incorporated by reference. Once sugars are fermented into a liquid biofuel they may be upgraded to a pure anhydrous fuel via conventional distillation and dehydration processes.

Recovery of the sugars from the biomass residue is preferably achieved through solid-liquid separation. For example, as shown in FIG. 2, a solid/liquid separation step 18 may be used to recover the sugars from the biomass residue. Solid/liquid separation may be performed using a number of methods including, but not limited to, centrifuging or pressing. Preferably, pressing may be accomplished with a hydraulic press. However, numerous types of mechanical or machine presses may be used. For example, a mechanical press such as a conventional screw press, a hydro-mechanical press, a pneumatic press or any other type of press that can apply the necessary pressure to remove the hemicellulose hydrolysate from the cellulose/lignin residue may be used. The press may have a range of capabilities and configurations for pressing out the hemicellulose hydrolysate. Preferably the press can generate from at least about 10.5 kg/cm² to about 21.1 kg/cm². In other embodiments, it is desirable if the press can generate at least approximately 1,410 kg/cm².

Pressing has additional advantages because the biomass residue (which will comprise cellulose and lignin at this point) may be more valuable as a coal replacement if its density can be maximized and its moisture content minimized, thereby increasing its energy density. For pulp mill feed there are no requirements for moisture or density but minimization of fiber damage is important. Pulp quality is measured based on its fiber length, among other variables, but not moisture content. However, if a high energy density fuel replacement is made instead of paper pulp, reducing the moisture content is an important factor.

Accordingly, the final product that the biomass residue is to eventually be used for may determine what size and kind of press to use for solid/liquid separation. For example, if the biomass residue is to eventually be used to generate cellulose and/or lignin fibers to make paper products, cardboard, or fiberboard, a lower pressure, such as in the range of 10.5 kg/cm² to 21.1 kg/cm² may be advantageous to minimize damage to the cellulose fibers. In processes that turn the biomass residue into high energy density fuel, higher pressures may be used to minimize the moisture content, without regard to fiber quality. As a result, it may be desirable to employ pressures of about 1,410 kg/cm² or even higher. In other embodiments, however, pressures within the range of 10.5 kg/cm² to 21.1 kg/cm² may still be used, as presses generating these types of pressures are readily available and comparatively inexpensive as compared to presses that are capable generating about 1410 kg/cm² of pressure. For example, presses that generate between about 10.5 kg/cm² and 21.1 kg/cm² of pressure are routinely used in the wine and olive oil industries to press grapes and olives, respectively.

When sugarcane bagasse is used as the biomass from which the hydrolysate is pressed, fiber condition will generally be unimportant unless the biomass residue will be used as a feedstock for a paper mill. However, when the biomass residue derived from bagasse is used as a high energy density fuel replacement, the moisture content is an important factor. Therefore, higher, rather than lower pressures, may be desirable for purposes of performing the solid/liquid separation step 18.

Pressing is also advantageous because it reduces dilution from wash water prior to solid liquid separation. Wash water may be used to help separate the hydrolysate from the biomass. However, wash water will dilute the sugar stream and thus lower the resulting ethanol concentration in the fermented hydrolysate. If wash water is used, dilution of the sugar stream may be mitigated by the use of evaporators or similar machinery to reduce water content in the hydrolysate. The recovered water from evaporation may be recycled into subsequent wash processes. The addition of evaporation as a process step increases the sugar concentration of the hydrolysate and the ethanol concentration resulting from fermentation and thereby reduces the costs of distillation.

Once the monosaccharides are separated from the biomass, there are a number of microbes that may be used for converting the monosaccharides of the biomass hydrolysate into ethanol or other biofuels in fermentation step 108. For example, if the biomass hydrolysate comprises a cellulose hydrolysate, so as to include glucose (which is a hexose), the glucose in the hydrolysate may be fermented by a number of yeast strains including Saccharomyces cerevisiae (traditional baker's yeast) and Kluyveromyces marxianus to name a few.

On the other hand, if the biomass hydrolysate comprises a hemicellulose hydrolysate, the hydrolysate will include the pentoses xylose and arabinose, and a lower concentration of hexoses, except in the case of softwood hydrolysate. In the case of softwood hemicellulose, the hexose mannose is the major saccharide and the pentose xylose is the next most abundant. Microbes that can convert the combination of pentoses and hexoses found in hemicellulose hydrolysate into biofuels, such as ethanol, are not as abundant as those available for cellulose hydrolysate. To convert sugars from hemicellulose hydrolysate into ethanol, microbes that can ferment both five-carbon and six-carbon sugars are preferably utilized so that all of the available constituent sugars of the hemicellulose hydrolysate may be converted to ethanol or other biofuels. The same is true if the biomass hydrolysate comprises a combination of cellulose hydrolysate and hemicellulose hydrolysate. Microbes that can ferment hexoses and pentoses may be derived from the genera Pachysolen, Kluyveromyces, Pichia, and Candida. Pachysolen tannophilus is preferably used in fermentation of a liquid hydrolysate comprising a hemicellulose hydrolysate. In particular, when immobilized, Pachysolen tannophilus has been found to effectively ferment hemicellulose hydrolysate produced from softwood.

In addition to immobilized yeasts, immobilized bacterium may also be used to ferment hexose and pentose sugars in biomass hydrolysate. For example, the recombinant bacterium Zymomonas mobilis (NREL recombinant 8b) may be used to ferment hemicellulose hydrolysate produced from softwood, hardwood, and/or herbaceous sources.

Microbes with complementary metabolic properties may also be combined in the same fermentation process in step 108 to allow their complementary properties and abilities, such as complementary hexose and pentose fermentation capabilities or complimentary metabolic rates, to be used together. For example, because recombinant Zymomonas is unable to ferment mannose, the most prevalent sugar contained in softwood hydrolysate, the recombinant Zymomonas mobilis is preferably paired with a complementary yeast or bacterium that is able to effectively ferment the hexose mannose to ethanol or another biofuel when it used to ferment softwood hydrolysate. On the other hand, in the case of sugarcane bagasse, where the hydrolysate primarily comprises xylose and glucose, another microbe is not required to assist the recombinant Zymomonas to achieve a satisfactory fermentation of the contained sugars.

Other combinations of microbes are also possible including pairing different bacterium together, pairing different yeasts together, pairing various yeasts and bacterium together, or pairing or combining any number of microbes with complimentary features including using any number of microbes at the same time. As the number of combined microbes increases, however, their capabilities may begin to overlap significantly and thereby reduce the additive value of the additional microbes.

Depending on the biomass and treatments employed, the pretreatment and hydrolysis step 16 may yield soluble sugars from the biomass in the form of xylose, mannose, arabinose, galactose, and glucose ready for fermentation in step 108. However, other secondary products, which are inhibitory to the fermentation step 108, are also produced or extracted from the biomass. The concentrations of fermentation inhibitors that form in converting biomass to fermentable hexoses and pentoses will vary depending on the source of the biomass and the methods used for the pretreatment and hydrolysis step 16. For example acetic acid is produced by cleavage of acetyl groups from hemicellulose. In addition, some of the pentoses and hexoses are degraded due to dehydration into furfural and HMF. Phenolic and polyphenolic compounds (collectively “Phenolic Compounds”) are also formed from the degradation of lignin. While the generated Phenolic Compounds, furfural, HMF, and acetic acid are all potentially valuable compounds, they are also fermentation inhibitors, and may prevent or inhibit fermentation, particularly as their concentrations increase.

In addition, furfural and HMF degrades to produce levulinic acid, acetic acid, and formic acid, which are even more potent fermentation inhibitors. Phenolic and polyphenolic compounds produced from hydrolysis of wood hemicellulose and the concomitant lignin degradation include guaiacol, vanillin, phenol, vanillic acid, syringic acid, salicylic acid, gentisic acid, and others. Many of these compounds, for instance vanillin and vanillic acid, are known to inhibit the growth of and/or fermentation with microbial yeasts, such as Pachysolen and Saccharomyces.

In addition to secondary products made from the degradation of hemicellulose components, other molecules may be extracted from biomass by the pretreatment and/or saccharification conditions during the pretreatment and hydrolysis step 16. These extracted compounds may include terpenes, sterols, fatty acids, and resin acids. These extracted compounds can also be inhibitory to metabolic processes, including fermentation, in yeast and other microbes, such as bacteria.

Furthermore, metal cations including calcium, aluminum, potassium, and sodium are found in hemicellulose hydrolysate and heavy metals may be present from degradation of the metal vessels due to hydrolysis. The presence of such metal cations may also be inhibitory above certain concentrations.

As made clear from the foregoing discussion, the environment experienced by microbes in biomass hydrolysate is in stark contrast to a defined, artificial medium where all or most of these additional inhibitors are not present or are added experimentally one at a time to study their effects. Indeed, in a biomass hydrolysate the various inhibitory compounds discussed above, as well as others, may work synergistically with one another so that a non-inhibitory amount of a certain compound may become inhibitory in the presence of one or more additional compounds that are also below their respective individual inhibitory concentrations.

Because many secondary products can degrade the fermentation process as their concentrations increase, prior methods for conversion of biomass into ethanol have employed a costly conditioning step to remove or reduce the concentration of inhibitors from the hydrolysate prior to fermentation. Furfural, HMF, and acetic acid, as well as phenolics are the most commonly found inhibitors in biomass hydrolysate. Levels in the range of 0.2-5.0 g/L furfural, 0.2-6.0 g/L HMF, and 3.0-10.9 g/L acetic acid are considered common and may greatly reduce fermentation or prevent it all together. Likewise, concentrations of phenolics in the range of 0.1-10 g/L are common and may be inhibitory. A method commonly used to ameliorate the toxicity of hydrolysates by reducing HMF and furfural concentration is pH adjustment through “overliming” with calcium hydroxide. Overliming is the process whereby lime is added beyond that necessary for pH adjustment. Even after overliming, however, high levels of inhibitors may still exist. In addition, overliming precludes recovery of secondary products that have high value from the hydrolysate.

In order to deal with the potential for high levels of inhibitory secondary products often found in biomass hydrolysate—for example, levels that would inhibit the fermentation microbes in their free state—during the fermentation step 108, the fermentation microbes may be immobilized, and more preferably immobilized in calcium alginate. Immobilization confers an increased resistance of microbes to inhibitors and therefore, increases fermentation efficiency. For example, immobilization in calcium alginate greatly reduces the susceptibility of the yeast Pachysolen tannophilus to inhibitors contained in softwood hydrolysate. Preferably the calcium alginate, or other material used to immobilize the microbes, is in a form with a high surface area such as in a bead, sponge, or mesh form.

Immobilization of microbes is the attachment or inclusion in a distinct solid phase, such as calcium alginate, that permits exchange of substrates, products, inhibitors, etc. with the microbe, but at the same time separates the microbes from the bulk biomass hydrolysate environment. Therefore, the microenvironment surrounding the immobilized microbes is not necessarily the same as that which would be experienced by their free-cell counterparts. As a result, for example, the present patent document teaches processes for immobilizing Pachysolen tannophilus and for fermenting pentoses and hexoses in the presence of inhibitors found in hemicellulose hydrolysate, even at concentrations that would inhibit the fermentative microbe in its free state.

By immobilizing the fermentative microbe(s) during the fermentation step 108, the need for conditioning the biomass hydrolysate to reduce the concentration of, or possibly even completely remove, inhibitory secondary products is significantly ameliorated. This is because the need to lower the concentration of inhibitory secondary products to the levels necessary for fermentation using free microbes is eliminated. Thus, as reflected in FIG. 2, conditioning step 22 to reduce the concentration of inhibitors is an optional step.

Conditioning the biomass hydrolysate in conditioning step 22 to reduce the concentration of inhibitory secondary products may still be desirable where, for example, the concentration of the secondary products (either individually or in combination) is sufficiently high to interfere with the fermentation of sugars even by the immobilized microbe(s). In such cases, however, the concentration of the inhibitory secondary products will generally not need to be reduced to the same levels as necessary for fermentation using free microbes and thus a less severe and less costly conditioning process may be employed. To offset the costs associated with the overall fermentation process, it may also be desirable to recover secondary products having a high value through an optional high value secondary product recovery step 24 shown in FIG. 2. Following partial removal (and possible recovery) of many secondary products from the biomass hydrolysate, however, the concentrations of these products may remain sufficiently elevated within the hydrolysate, particularly considering the synergistic nature of the inhibitors, to interfere with fermentation of sugars to ethanol or other biofuel by the fermentative microbe(s) in their free state. Accordingly, the use of immobilized fermentative microbe(s) in fermentation step 108 may be very beneficial even when the optional conditioning step 22 is employed to reduce the concentration of secondary products contained in the biomass hydrolysate.

In some instances, it may also be desirable to perform conditioning step 22 even when the concentration of inhibitory secondary products is insufficient to inhibit fermentation by the immobilized microbe(s) where, for example, the secondary products have high value and thus it is desirable to separately recover the high value secondary products through high value secondary product recovery step 24. This may be desirable, for example, where the net value of the recovered high value secondary products may be used to offset, and hence lower, the costs associated with the overall fermentation process.

There are numerous methods of performing the conditioning step 22 to reduce the concentrations of inhibitory secondary products. Employing different conditioning methods for conditioning step 22 will result in different concentration levels of inhibitory secondary products remaining in the hydrolysate. The method of conditioning chosen for conditioning step 22 may depend on a variety of factors, including the sensitivity of the microbe used during fermentation to inhibitory secondary products, costs, and whether there is a desire to recover high value secondary products during a recovery step 24. The more sensitive the microbe, the more desirable it will be to reduce the concentration of the inhibitory products from the biomass hydrolysate during conditioning of the hydrolysate in step 22. Immobilization of the fermentative microbe(s), however, will decrease the sensitivity of the microbe to inhibitory secondary products and thus may reduce the complexity and costs incurred during conditioning step 22. Some of the conditioning methods that may be employed in conditioning step 22 to reduce the concentration of secondary products include, but are not limited to: 1) overliming of hydrolysate; 2) activated carbon (AC) treatment followed by pH adjustment; 3) ion exchange followed by overliming; 4) AC treatment followed by ion exchange; and 5) AC treatment followed by nanofiltration.

During the conditioning process 22, the inhibitory secondary products, which have value when isolated, may be recovered as further shown in FIG. 2 as optional step 24. Secondary products may be recovered in step 24 from the hydrolysate and subsequently used for other purposes. Hydrolysate from solid-liquid separation 18 contains a number of high value secondary products including, but not limited to the mineral acid used in the pretreatment process 16, such as sulfuric acid, acetic acid hydrolyzed from hemicellulose polymers, anti-oxidant molecules (phenolic and polyphenolic compounds) liberated from the partial hydrolysis of lignin, other organic acids, nutraceutical, cosmeceutical, or pharmaceutical products, and different furans and furan derivatives, such as 5-hydroxymethylfurfural and furfural. High value secondary product recovery 24 may be accomplished by adsorption to different matrices, including activated carbon, ion exchange resin, ion exchange membrane, organic molecule “scavenging” resins, polystyrene beads, or another such medium with a high surface area. High value secondary product recovery 24 may also be accomplished by separating them from soluble hexoses and pentoses through ion exclusion chromatography, pseudo-moving bed chromatography, high performance liquid chromatography or by filtration methods including micro-, nano-, and ultrafiltration using hollow fiber or membrane technologies. High value secondary product recovery 24 may include several of the aforementioned processes in series to recover different molecular species. Furthermore, the recovery processes 24 may be tailored to recover specific secondary products according to the nature of the starting biomass. Recovery of high value secondary products 24 also provides a benefit to the fermentation process 26, as many of the recovered secondary products (acetic acid, furans and their derivatives, phenolic and polyphenolic compounds, levulinic acid, formic acid, and others) are inhibitory to yeast and bacterial fermentation of sugars to ethanol. Thus, recovery of high value secondary products 24 both increases the economics of the entire process and allows for more efficient fermentation 26 of the pentoses and hexoses.

Even following partial recovery of many high value products 24, the concentrations of these products may remain elevated, and considering the synergistic nature of the inhibitors, are sufficient to interfere with fermentation 26 of sugars to ethanol. In order to more efficiently ferment the hexoses and pentoses that have been separated from the biomass residue and high value products, the fermentation microbes may be immobilized. Immobilization confers an increased resistance of microbes to inhibitors and therefore, increases fermentation efficiency. For example, immobilization in a calcium alginate greatly reduces the susceptibility of microbes, such as the yeast Pachysolen tannophilus, to inhibitors contained in softwood hydrolysate. Preferably the calcium alginate, or other material used to immobilize the microbes, is in a form with a high surface area such as in bead, sponge, or mesh form.

In general, microbes may be immobilized for fermentation of biomass hydrolysate in step 108 using a number of different methods. Microbes may be bound to a matrix material or, more preferably, immobilized by entrapment in the matrix material. For example, microbes may be immobilized by entrapment using a drop-forming procedure. The resultant beads may be of different size and possess different pore sizes. For example, the beads may range in size from 0.1 mm to 5 mm in diameter, more preferably the beads may range from 2 mm to 3 mm in diameter, and more preferably the beads are about 3 mm in diameter.

The drop-forming procedure may be enhanced through a number of processes. The beads, may be hardened to different degrees and may have coatings applied to withstand shear forces in a reactor and to reduce cell loss. For example, if calcium alginate is used, the beads may be dried to increase compression stress. The beads may also be hardened by glutaraldehyde treatment or coated with catalyst-free polymer to enhance their stability. The beads may be recoated with plain alginate as a double layer to enhance their gel stability. Furthermore, the beads may have a polyacrylamide coating to enhance their structural stability. The beads may also be coated with a copolymer acrylic resin to increase diffusion and reduce cell leakage. Similarly, other additions to the drop forming procedure may be incorporated to enhance the effectiveness of the matrix.

Other techniques for improving the efficiency of immobilized microbes include increasing the surface area of the microbe/immobilization medium mixture once it is formed. For example, a Pachysolen tannophilus/calcium alginate or other microbe/calcium alginate mixture may be applied as a coating to a natural or synthetic, high surface area, support structure. In one implementation, the support structure only need be able to support the microbe/immobilization medium and itself. For example, the support structure may comprise a ceramic sponge, honeycomb, reactor packing material or other support structure to increase the surface area per mass of the microbe/immobilization medium when it is applied. The mixture may also, or in the alternative, be applied to parts of the reactor surfaces, such as, the walls or the surface of the mixing devices.

In addition to immobilization by entrapment, the microbes may be immobilized by other methods including adsorption, cross-linking, or immobilized by any other means capable of providing a micro-environment for the microbe.

A variety of different materials may be used to immobilize microbes. If the microbes are immobilized using entrapment calcium alginate, a natural product from brown algae (seaweed) may be preferably used. However, other materials, both natural and synthetic, may also be used to immobilize microbes using entrapment including carrageenan, xanthan gums, agarose, agar and luffa, cellulose and its derivatives, collagen, gelatin, epoxy resin, photo cross-linkable resins, polyacrylamide, polyester, polystyrene and polyurethane.

Other materials that may be used to immobilize microbes using adsorption or other immobilization methods include kieselguhr, wood, glass ceramic, plastic materials, polyvinyl acetate, and glass wool.

When combining microbes with complimentary properties, the microbes may be combined within the same immobilization vehicle, or the microbes may be immobilized separately and the separately immobilized microbes combined in the same fermentation reactor. For example, if calcium alginate beads are used as the immobilization vehicle, different complimentary microbes may be combined within the same bead. As one example, to effectively ferment softwood hydrolysate, which contains the sugars mannose, galactose, glucose and xylose, to ethanol, one may combine Zymomonas mobilis, NREL strain 8b, which ferments glucose and xylose to ethanol, with Saccharomyces cerevisiae, which ferments mannose and galactose, into a single bead product. In this way advantageous fermentative properties of different microbial species are combined in a single bead product.

Alternatively, separate beads can be made containing each microbe and then the beads may be combined in the fermentation reactor. For example, the fermentation of the hexoses and pentoses to fuel may be performed by combining beads composed of different microbial species with complementary hexose and pentose specificities, metabolic rates, or the like. In yet another example, different microbes are immobilized in separate reactors and the biomass hydrolysate is then run through each reactor to expose the biomass hydrolysate to each microbe. In addition, different immobilization methods may be combined with different microbes.

One of the many advantages of immobilizing the microbes is that the microbes become more stable and bioreactors may be run in a continuous mode instead of batch mode. Running the bioreactor in a continuous mode is advantageous for efficiency reasons but the microbes may begin to lose metabolic efficiencies after long periods of use. In order to restore metabolic efficiency, the immobilized microbes may be periodically treated with yeast growth medium. For example, the Pachysolen tannophilus immobilized in calcium alginate may be periodically treated with a yeast growth medium to restore metabolic efficiency.

Another advantage of microbe immobilization is that microbe biomass may be better retained within a continuous fermentation reactor. In a continuous fermentation involving a high flow rate, such as that which is experienced during the continuous running of a columnar up-flow reactor, free cells will tend to wash out, thereby lowering the fermentation rate of the fermentation reaction. Immobilization reduces or prevents wash out in high flow rate continuous flow reactors.

Another advantage of immobilizing microbes is the ability to obtain a high biomass concentration in a continuous fermentation process. In a column upflow reactor, as a non-limiting example, more than half, preferably about two thirds to about three quarters of the reactor volume will be composed of the bead material and the rest will be inter particle void volume when the fermentative microbes are immobilized in beads of about 2 mm to 3 mm in diameter. In the case of using yeast as the fermenting microbe, where 5% of the volume of the bead is yeast biomass, the reactor will effectively contains about 3.3 to 3.75% by volume yeast biomass, which is a relatively high yeast concentration for a fermentor.

Other benefits of yeast and bacteria immobilization by entrapment in calcium alginate over free cells in suspension include greater ethanol tolerance, possibly due to changes in cell membrane composition; greater specific ethanol production, increased rate of ethanol production due to increased glucose uptake and lower dissolved CO₂ in solution, and increased thermo-stability of bacteria.

As described above, there are numerous methods of actually immobilizing the microbes. In one preferred embodiment for immobilizing Pachysolen tannophilus in calcium alginate, the microbes are initially immobilized in sodium alginate which is then converted to calcium alginate. Sodium alginate can have different viscosities when a given amount is dissolved in an aqueous solution. Viscosities for different sodium alginate products range from 100 or 200 mPa, to even as much as 1236 mPa. In a preferred embodiment, alginate with medium-low viscosity of about 324 mPa is used to produce beads, although alginates with different viscosities may be used for different biomass hydrolysates or for solid-state ferments.

The sodium alginate is prepared by adding from 0.05 to 10%, or preferably about 3.5% (w/v) sodium alginate to deionized water. Alternatively, the sodium alginate can be dissolved into growth medium, into a mixture of vitamins, including biotin, or into growth medium supplemented with vitamins, or into a natural solution containing biotin. The initial sodium alginate concentration will depend on the final concentration desired to produce beads and on the volume added by mixing with a concentrated microbe slurry.

In order to get some sodium alginate preparations into solution, the mixture may be heated and stirred on a stir plate. This method is appropriate for producing smaller laboratory volumes of sodium alginate, but less attractive for large volumes. Furthermore, heating alginate polymers may cause some amount of hydrolysis of the alginate and thereby change the properties of the alginate solution, including its viscosity. As a result, it may be desirable to use a sodium alginate preparation that does not require heating in order to go into solution. In embodiments where the alginate may not be heated for solubilization nor autoclaved for sterilization, it may be desirable to treat the alginate with a chemical sterilizer or it may be desirable to irradiate the alginate with ultraviolet light for sterilization.

Cells may be cultivated in their respective media, and pelleted by centrifugation. Alternatively, a mass of Pachysolen or other in fermentative microbe may be propagated in at least a 10 L, or more preferably at least a 200 L, or even more preferably at least a 2000 L bioreactor to a concentration of about 1 to about 20 grams wet mass per liter growth medium. The resulting biomass may then be concentrated using, for example, a tangential flow filtration device to produce a 20-70% wet mass slurry of Pachysolen cells. This technique is particularly well suited for the production of large volumes of calcium alginate beads having one or fermentative microbes, such as Pachysolen, immobilized therein.

Following concentration, the concentrated cells are then recovered and thoroughly mixed with the sodium alginate medium. Mixing the alginate with the microbial cells can occur in the same device as is used for the resuspension of the alginate or in a separate device. The mixing continues to homogenity of the mixture. Mixing of the microbes with the highly viscous sodium alginate solution requires a mixing method that does not shear the microbes, such as a reciprocating disc mixer. The cell loading into the sodium alginate medium is both organism and substrate dependent. For example, a suitable target loading for Pachysolen tannophilus in hydrolysate is at least 5 g cells/100 mL sodium alginate medium.

Calcium alginate beads are produced by extruding the sodium alginate medium/cells into a sterile calcium chloride solution. A peristaltic pump and sterilized Master-flex Bulk-Packed Silicone Tubing that has an attached sterile 18 G needle may be used in the extruding process. The entire process is preferably done aseptically. In an alternative embodiment that is more suitable where large amounts of immobilized microbe beads are desired to be produced, a sterile 96 hollow 19 gauge pin device may be used in place of an 18 gauge needle. The beads may then be produced by extrusion and gravity dropping. Other methods may include a so-called Jet Cutter to produce beads from a continuous stream of an alginate/microbe slurry. Other modifications of producing beads from a continuous stream include using electrostatic attraction to produce droplets, using vibration to produce droplets, using air to produce droplets, and using a rotating disk atomizer, to name a few.

In order to exchange sodium ions with calcium ions to effect polymerization of the alginate, beads are dropped in a solution containing calcium chloride. In one method, a 0.22M solution of calcium chloride dihydrate is also prepared in deionized water to receive sodium alginate/microbe mixture. The sodium alginate medium and calcium chloride solution may both be autoclaved for sterilization purposes. The beads may be kept at 4° C. in the calcium chloride solution for about 60 minutes to harden. Once the beads have hardened, they are preferably rinsed several times with sterile deionized water. In a preferred embodiment, the beads are dropped into sterile growth medium containing 0.1 to 0.25 M calcium chloride. The growth medium may also contain different vitamins or biotin. After about 30 minutes of hardening, the beads may be either used immediately in a fermentation or may be stored at 4° C. until use. There is no need to rinse beads prior to use or prior to storage when hardening is carried out in such a growth medium.

In certain implementations, it may also be desirable to recycle components of the immobilization processes. The solid calcium alginate used to immobilize microbes in beads or on a support structure may delaminate, break-up, shear, or otherwise physically degrade after prolonged use. In addition, the microbe/calcium alginate mixture may also become degraded and discolored through repeated use due to the trapping of contaminants such as extractives, microbial inhibitors, and other materials. Degradation of the structure, whether due to physical and/or chemical degradation affects the performance of the fermentation process. To overcome deleterious effects of this degradation, new or fresh microbe/calcium alginate mixture may be used in the bioreactor to improve the reactors performance. However, the frequent replacement of the mixture may be uneconomical both in terms of the material costs associated with production of the calcium alginate, but also due to the cost of the lost microbes.

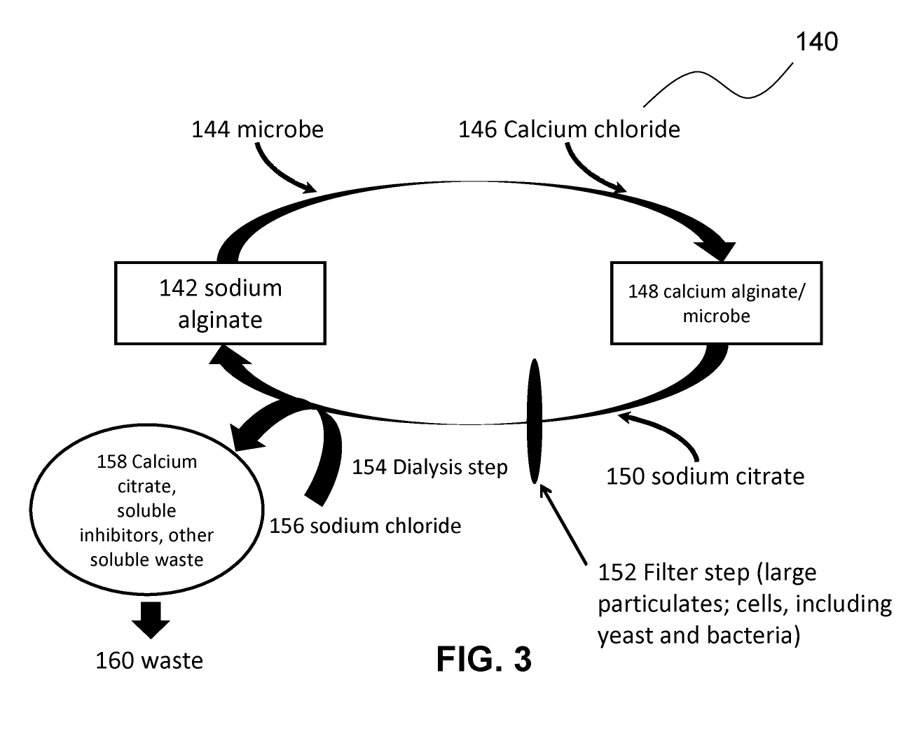

FIG. 3 illustrates a process 140 for recycling calcium alginate used in the microbe immobilization process. For example, in the case of Pachysolen tannophilus immobilized in calcium alginate beads, the calcium alginate from the beads used to immobilize the microbes may be recovered and recycled using process 140. In process 140, the degraded microbe/calcium alginate mixture 148, is dissociated with a calcium chelator complexed with a monovalent ion 150, such as sodium citrate or potassium citrate. Step 150 of process 140 dissociates the alginate and liberates the microbes (bacteria or yeast cells). In one preferred embodiment of process 140, step 150 is accomplished by stirring the microbe/calcium alginate mixture in 20 g/L sodium citrate or potassium citrate with a pH 8.2. at room temperature for 15 minutes.

Once the microbes have been liberated and the alginate dissociated, the solution is filtered to remove the large particulate and microbes (bacteria or yeast cells) in step 152. The filtered solution is then dialyzed, step 154, against a sodium salt 156, such as sodium chloride, to remove the calcium citrate, extractives, and soluble microbial inhibitors 158. The resulting dialysis of the filtered solution with an inorganic salt, such as sodium chloride, regenerates sodium alginate. The toxic materials are removed as waste stream 160. The sodium alginate is concentrated during dialysis and then used again to produce calcium alginate in steps 142, 144, and 146 as described above. In one preferred embodiment, the sodium alginate is used to immobilize Pachysolen tannophilus in calcium alginate beads as taught in the above process.

Fermentation may occur using a number of methods. If the microbes are immobilized, the hemicellulose hydrolysate may be removed from the biomass residue, and fermented ex-situ. If the microbes are free, than fermentation may occur ex-situ or within the biomass residue. Although immobilizing the microbes is the preferred method of fermentation, immobilization is not required and the fermentation may be performed with ‘free’ microbes. One example of a free microbe that may be used to ferment hemicellulose hydrolysate is Zymomonas mobilis (NREL recombinant 8b). As mentioned above, Zymomonas mobilis (NREL recombinant 8b) may be used to ferment five-carbon and six-carbon sugars in solid state fermentation.

A variety of bioreactor designs, including a traditional non-stirred fermenter or stirred fermenter, may be used for the fermentation of the biomass hydrolysate using free or immobilized microbes. The reactor may be a submerged reactor or other type of liquid reactor. In order to provide the highest yield, a submerged reactor is preferable to ferment five-carbon sugars.

In the case of microbes that are immobilized, a packed bed reactor could be utilized, or a tankage system similar to that employed for carbon-in-pulp processes in the gold mining industry could be used. In the latter, beads would be moved counter-current to the solution flow and could be easily recovered for regeneration. Thin film reactors may also work well with immobilized microbes.

In addition, solid/liquid contactors may be used with immobilized microbes. These types of reactors include ion exchange columns, packed bed reactors, trickle flow reactors, and rotating contactors. Other reactors that may be used are fluidized-bed and upflow type reactors.

If the entrapment method of immobilization is used, the microbes may be incorporated into a bioreactor using a number of different methods. In addition to beads, the matrix/microbe gel may be applied to a support structures to increase the effective surface area. These configurations may include coating paddle structures, used in stirred tank reactors, rotating contactors, and thin film reactors. The microbes could also be incorporated in large three-dimensional open-cell supports for use in trickle flow reactors or fluidized-bed and upflow reactors.

Bioreactors based on immobilized microbes offer several advantages over ‘free cell’ systems. One advantage is the increased feasibility to employ a continuous fermentation system. Immobilization ensures no loss of cell mass, such as occurs with batch fermentation. Continuous fermentation decreases production down-time compared to batch fermentation.

Once the ethanol has been fermented in the bioreactor it may then be distilled. The biomass residue, which is now mostly devoid of hemicellulose, continues on to be processed into a high value product such as paper pulp and paper products using the normal pulp processing steps 104 as shown in FIG. 1 or into a solid biofuel product or paper mill feedstock as reflected in FIG. 2.

In addition to extracting the hemicellulose from the wood chips that are a part of the typical paper making process, the hemicellulose from the wood waste like bark and branches may also be converted into a biofuel such as ethanol. Typically, a pulp and paper mill has a boiler for hog fuel (wood waste) combustion and for the production of steam. The steam is used to help power the pulp or paper mill. The hog fuel may be bark, wood chips from other wood that the plant does not want in the particular paper, and slash (limbs, needles, leaves) from harvest.

According to one embodiment of the present invention, the hog fuel may be first processed for hemicellulose removal and then sent to the hog boiler. The hemicellulose from the hog fuel may then be converted into ethanol or other biofuel. In this embodiment, the hog fuel may be processed by itself or combined with wood chips used for making paper pulp. One advantage to using just the hog fuel for hemicellulose extraction is that it reduces the anxiety of pre-processing the chips that would ultimately become paper or paper pulp. Processing the hemicellulose of the wood waste into biofuel increases the energy production of the mill without affecting any of the materials used in the paper product.

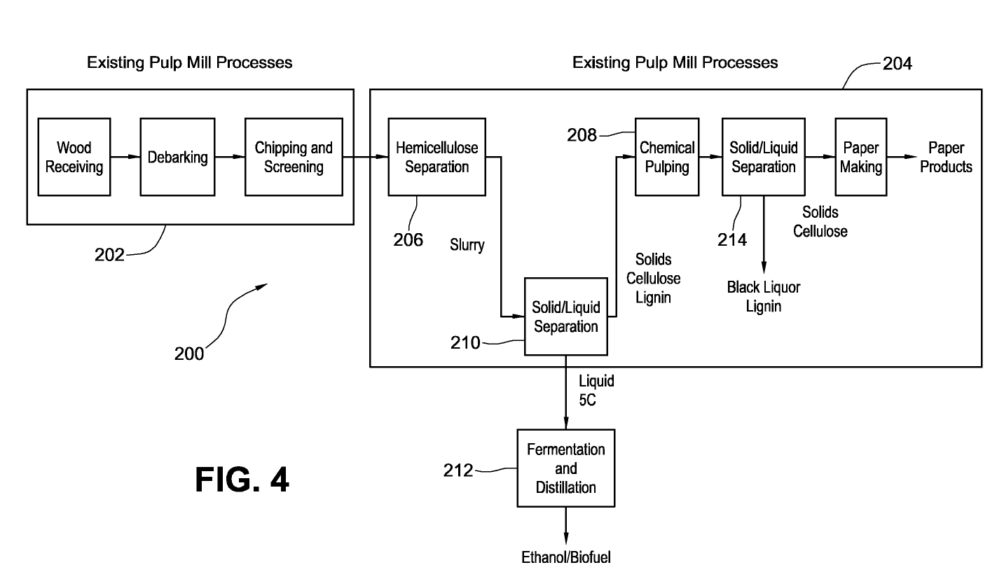

FIG. 4 illustrates a process for producing ethanol and paper pulp from wood. Process 200 is similar to process 100 except for the way the original equipment of the pulp mill is used. In process 100, illustrated in FIG. 1, new equipment is inserted into the pulp mill to remove the hemicellulose and ferment the sugars into ethanol or other biofuel. In FIG. 4, the original equipment of the pulp-mill is utilized to perform the hemicellulose separation 206 and removal 210. Fermentation is performed in new equipment in both process embodiments 100 and 200.

In process 200, the biomass may go through the same wood conditioning steps 202 as in process 100. Unlike in process 100, where the conditioned wood would be sent to newly added capital equipment, in process 200, the conditioned wood is sent to the same digester 206 it would normally be sent to in the paper pulping process. Instead of performing its normal function in the chemical processing of pulp, the digester in step 206 is used to separate only the hemicellulose.

The biomass and separated hemicellulose hydrolysate, which now contains the solubilized sugars, is then sent to the same liquid separator, in step 210, that would normally be used to separate out the black liquor. Instead, the solubilized sugars of the hemicellulose hydrolysate are removed and sent on to be ferment in the bioreactor in step 212. The remaining biomass residue is sent on to be processed into paper pulp in step 208.

In process 200, the same physical equipment is used in steps 206 and 208 and steps 210 and 214 respectively. By using the same equipment, existing pulp mills may be adapted to produce ethanol from hemicellulose with less capital investment.

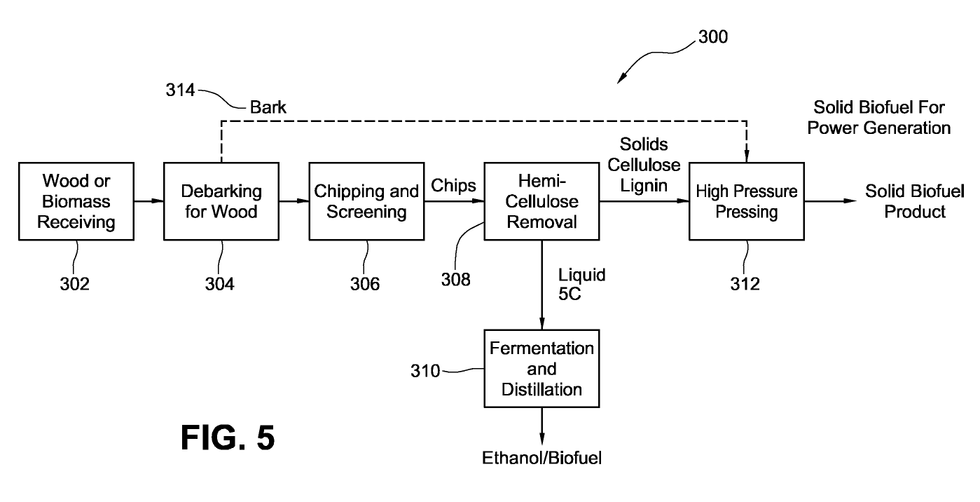

FIG. 5 illustrates a process for making ethanol and solid biofuel. Another alternative to the pulp mill add-on scenario would be to utilize the embodiment shown in FIG. 5 as a stand-alone system capable of treating forest materials to produce both ethanol and a solid lignin cellulose product suitable as a coal replacement for steam or power generation, or alternatively a pulp ready feedstock. This embodiment of the process could also be installed at existing forest product sites where several key process unit operations already exist, such as the wood yard and the debarking and chipping systems. The process could treat waste products such as saw dust or fresh wood chips. This process could also be applied to other herbaceous plants, such as switchgrass and Miscanthus, and to agricultural residue, such as corn stover and cobs and sugarcane bagasse.

As shown in FIG. 5, the wood conditioning steps 302, 304, and 306 are the same as in process 100 and 200. Also similar to process 100 and 200, process 300 hydrolyzes the hemicellulose and then separates the hemicellulose hydrolysate from the biomass reside in step 308. The five and six carbon sugars found in hemicellulose hydrolysate are then fermented and distilled into ethanol or other biofuel in step 310. However, in embodiment 300 the biomass residue, which includes cellulose and lignin, is put through a high pressure press and sold off as a solid biofuel product, instead of being sent on to be transformed into paper pulp. Alternatively, the solid biofuel product could be sold as a pulp ready feedstock to paper mills.

As mentioned above, the five and six carbon sugars that are solubalized from the hemicellulose may be separated from the biomass by using a press in step 308. The same press or even the same pressing step may be used to compress the cellulose and lignin in step 312.

Rather than making a paper pulp, process 300 creates a high energy density biofuel from the cellulose lignin residue. The pressed cellulose and lignin residue is an advantageous product for a number of reasons. This product has value not only as a fuel replacement but for resale in the paper pulping industry to be further processed into paper. Using a press reduces the amount of wash water needed and therefore, increases the ethanol concentration and energy density of the pressed biofuel. In addition, the low moisture content in the cellulose/lignin residue increases the energy density of the product and makes it more efficient to transport.

Table 2 lists typical ranges of hemicellulose, cellulose, and lignin in wood. Table 3 lists the typical relative energy densities of each.

TABLE 2
Typical Ranges of Wood Components

| | Softwood % | Hardwood % |
|---|---|---|
| Hemicellulose | 20-32 | 15-35 |
| Cellulose | 40-44 | 40-44 |
| Lignin | 25-35 | 18-25 |

TABLE 3
Typical Energy Densities Btu/lb

| | |
|---|---|
| Wood | 8600 |
| Lignin | 11,479 |
| Hemicellulose | 6,717 |
| Cellulose | 6,704 |
| Ethanol | 76,330 Btu/gal |

As an optional step in process 300, the removed bark from step 304 may be pressed back into the cellulose lignin residue in step 314 to become part of the solid biofuel product.

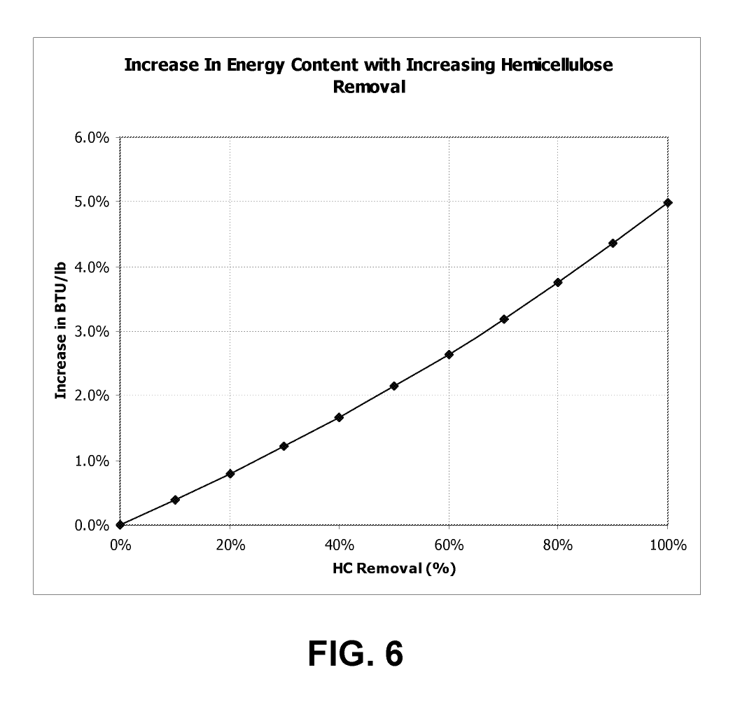

Because hemicellulose makes up between 15% and 35% of the wood source, and the energy density of hemicellulose is lower than the combined energy density of the other wood components, removing the hemicellulose will increase the overall energy density of the cellulose/lignin residue. The energy that is removed from the wood through hemicellulose removal is retained in the eventual ethanol product. FIG. 6 illustrates the energy density increase in the biomass residue as the percentage of hemicellulose removal is increased for a given moisture content of 25%.

It may be seen from FIG. 6 that the energy density of the residue goes up from 0% to 5% as 0% to 100% of the hemicellulose is removed.

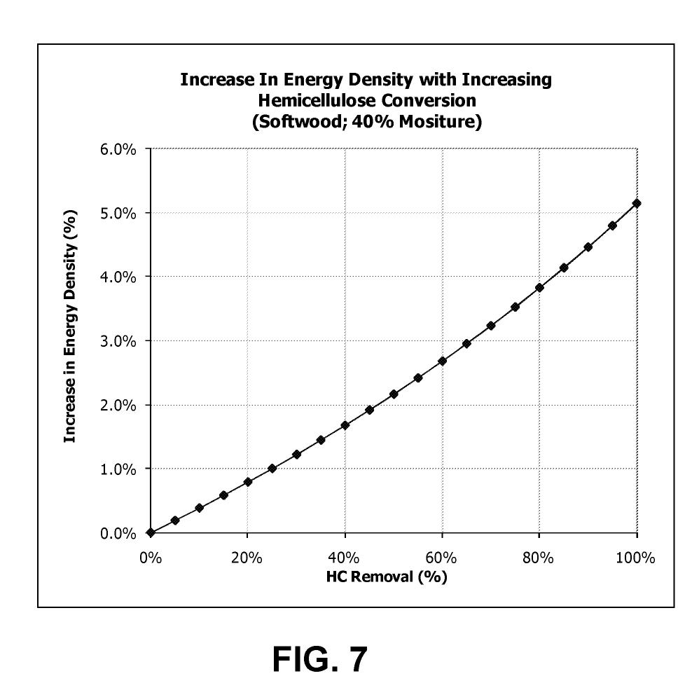

FIG. 7 illustrates the same principle as FIG. 6 but more specifically for pretreated wood residue with a constant 40% moisture content. Similar to FIG. 6, FIG. 7 demonstrates how as more hemicellulose is converted and removed the energy density of the residue increases because of the energy diluting effect of hemicellulose compared to lignin. The base energy density at 40% moisture and 75% HC conversion is 11.40 MJ/kg.

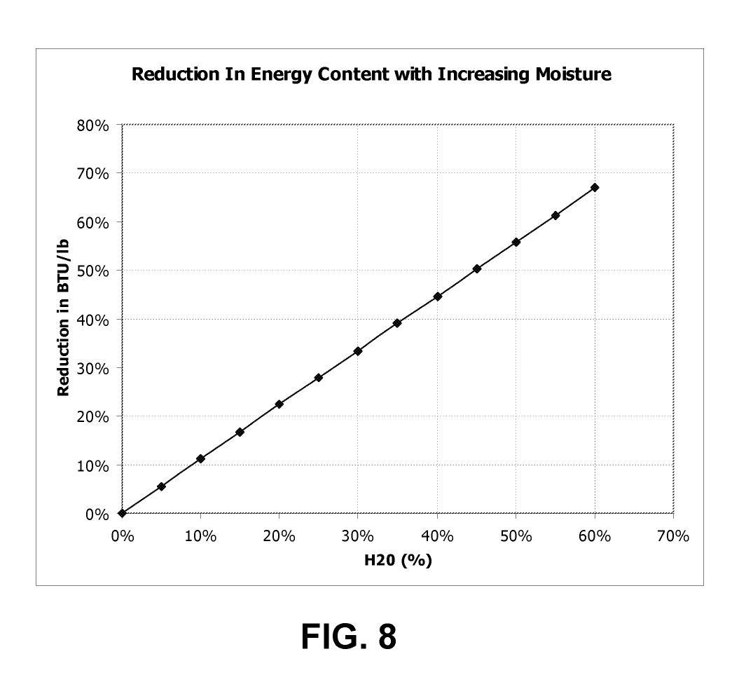

While hemicellulose removal will increase the energy density of the biomass residue, water plays a significant role in determining the energy density of the biomass residue. The biomass residue product may incur a 5% energy density increase due to hemicellulose removal. However, the biomass residue product has been reduced in weight 22.5% through water and volume reduction. Therefore, the energy density of the final pressed residue is higher than the original wood. FIG. 8 illustrates how the energy density of the residue changes as the percentage of water is reduced.

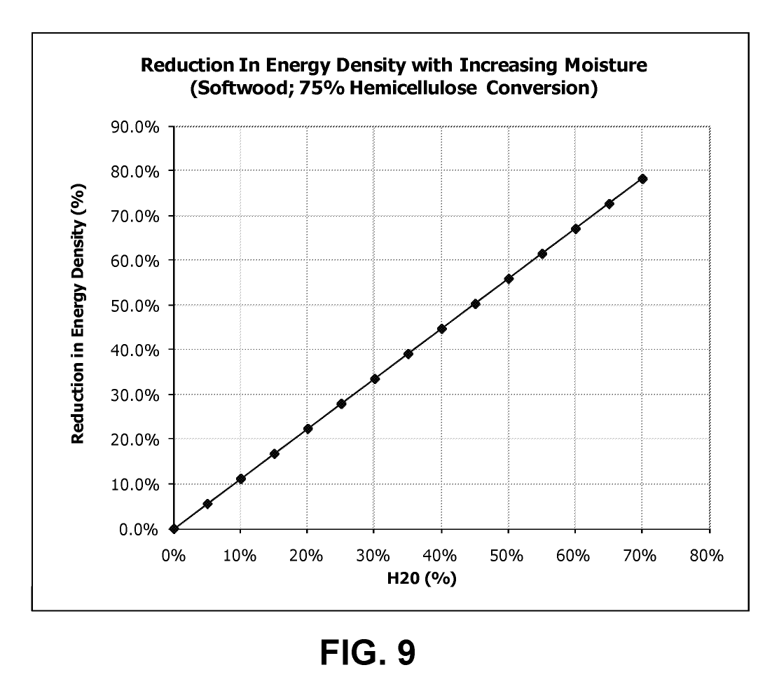

Similar to FIG. 8, FIG. 9 illustrates how for wood residue with 75% hemicellulose conversion, the energy density of the residue increases as the percentage of water is reduced. As moisture content increases, the energy density of the residue goes down. More water in the residue equates to more energy required to remove it in a boiler. The base energy density at 40% moisture and 75% HC conversion is 11.40 MJ/kg. Other biomass residues at similar levels of hemicellulose conversion show similar characteristics.

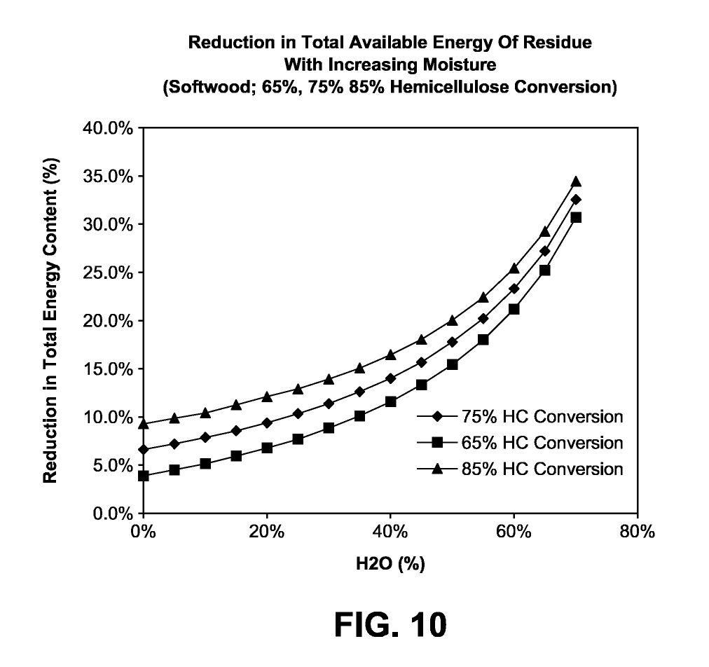

FIG. 10 illustrates the change in total available energy of the residue as compared to green wood over a range of moisture contents at three different levels of hemicellulose conversion (65%, 75%, and 85%). The base energy available from non-pretreated green wood at 50% moisture is 8.77 MJ/kg. The base for pretreated wood at 75% HC conversion and 40% moisture is 11.40 MJ/kg of residue which represents a reduction in total energy input into the boiler of 14.0%. This is due to the reduction in weight of the residue of 33.9%. 1 tonne of green wood (50% H2O) provides 8,766 MJ, while the pretreated residue from that wood provides only 7,541 MJ (40% H2O). Decreasing the moisture content reduces the energy loss impact from removing the hemicellulose. Energy density is often the most important factor not necessarily total energy content. Boilers operate on a fixed feed rate and a fixed flue gas flow rate. The lower the water and higher the energy density, the more energy that can be output from the boiler. Ultimately, more wood or wood replacement (gas or oil) needs to be burned to make up the energy loss.

Preferably the biomass residue product will have approximately 90% of the hemicellulose removed and a moisture content of about 25%. Other percentages of hemicellulose removal and moisture content may be achieved. Preferably, as much hemicellulose and as much moisture as possible is removed from the biomass residue product. This product is an attractive coal replacement or may be sold to paper mills to be further processed into paper.

One of the advantages of the processes in the present patent document is that the processes remove an otherwise low value product from the traditional wood pulping circuit to produce a high value liquid biofuel. Because pulp mills already have most of the equipment necessary for the extra steps involved in separating hemicellulose from biomass, the capital and operating cost of adding the capability to produce biofuel to existing plants may be lower than implementing other comparable wood-to-ethanol processes. The simplicity of the processes and the potential for pulping improvements make the processes very attractive to the pulping industry. In addition, the hemicellulose sugars are extracted as monomers and thus almost no enzymes are necessary.

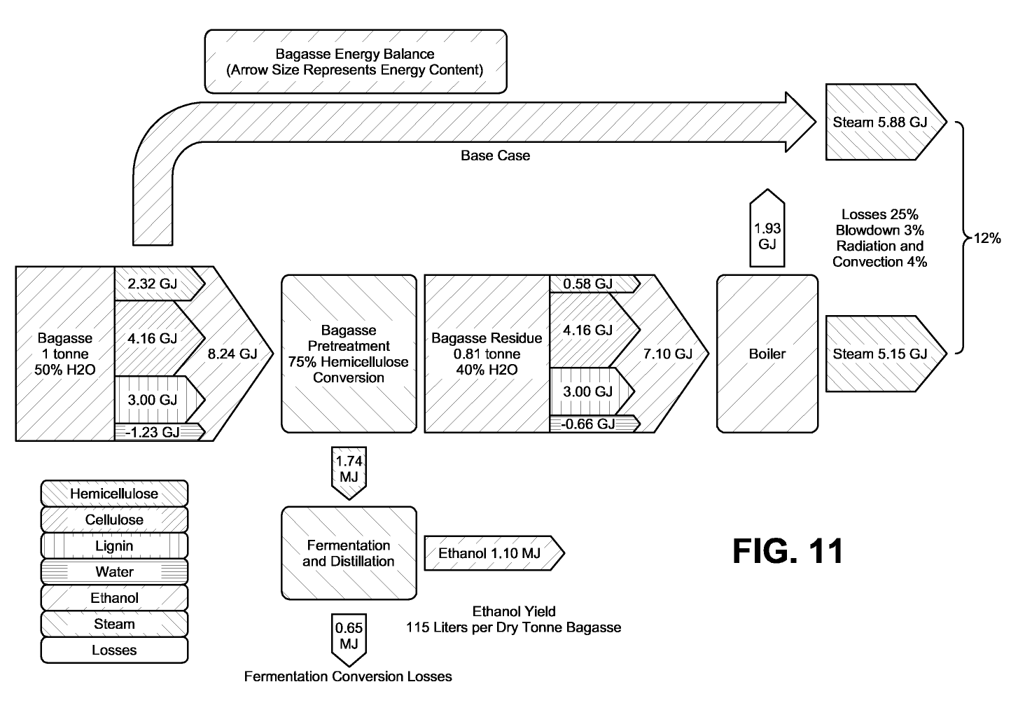

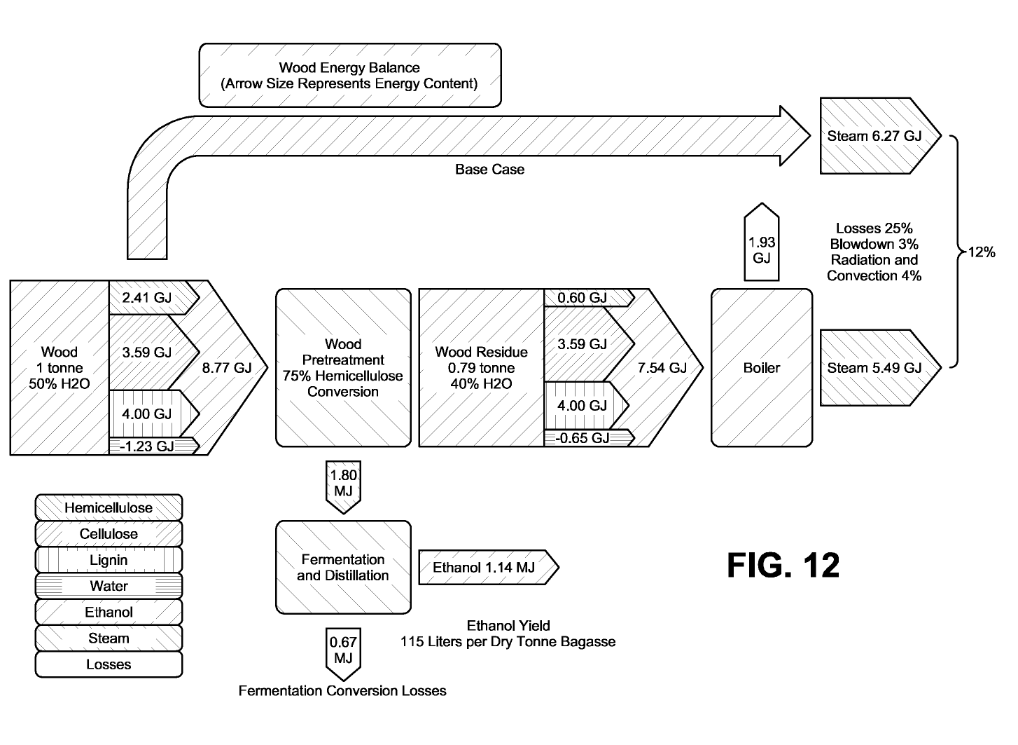

FIGS. 11 and 12 illustrates the energy flow for embodiments removing the hemicellulose and converting it into a biofuel such as ethanol and the energy flow without removing the hemicellulose for bagasse and wood biofuel sources respectively.

The following example demonstrates the application of one embodiment of the present patent document applied to beetle-killed pine. For the purposes of the present example Pachysolen tannophilus was either immobilized in calcium alginate beads with about a 3 mm diameter (generated using the method describe above) or was in a free cell state. Tables 4 and 5 below summarize the improvement of ethanol yield, and in glucose and xylose conversion resulting from the reactor design employed according to the present example.

The present example demonstrates the improvement of ethanol yield, and in glucose and xylose conversion, for calcium alginate-immobilized Pachysolen tannophilus in two different softwood hydrolysates ('A′ and ‘B’) over free (i.e. unrestricted) Pachysolen tannophilus. The hydrolysates were pH adjusted or overlimed and pH adjusted. The Pachysolen tannophilus strain NRRL Y2460 was used in carrying out the experiment; however, other adapted or mutated strains of Pachysolen tannophilus may also be immobilized in calcium alginate and used in processes according to the present patent document.

The pine was transformed into a softwood hydrolysate by dilute acid hydrolysis. The hydrolysate was either simply pH adjusted with sodium hydroxide or ‘overlimed’. As mentioned above overliming with calcium hydroxide is commonly used to ameliorate the toxicity of hydrolysates. The resulting solutions were fermented using Pachysolen tannophilus immobilized in 3 mm calcium alginate beads.

The beads were incubated in a flask of Yeast Peptone Dextrose (YPD) broth for 22 hours at 30° C. and 75 rpm. YPD is a standard yeast medium containing 10 g/L yeast extract, 20 g/L peptone, and 20 g/L dextrose. Similarly, the free cells were cultured from a working slant into a flask of YPD broth and incubated for 24 hours at 30° C. and 75 rpm.

To prepare the pH adjusted hydrolysate, the solution was adjusted to pH 6.0 with 8M potassium hydroxide, followed by filter sterilization. Preparation of overlimed and pH adjusted hydrolysate required overliming to pH 10.0 with calcium oxide, followed by a 30 minute hold at 50° C. under stirring conditions. The overlimed hydrolysate was then filtered to remove the solids. Following re-acidification to pH 6.0, the hydrolysate was filter sterilized.

Serum vials were aseptically prepared to obtain a final concentration of 95% hydrolysate with the following nutrient additions: 0.2% urea w/v, 0.2% yeast extract, and 0.05% potassium dihydrogen phosphate. The inoculation rate for immobilized beads was 0.2 g beads per mL. Following rinsing and re-suspension in sterile buffer, the free cells were inoculated at a rate of 0.3 OD₆₀₀ₙₘ per mL. All experimental conditions were set up in triplicate serum vials. The vials were aseptically vented and incubated for 72 hours at 30° C. and 75 rpm prior to sampling for analysis.

In pH adjusted hydrolysate “A”, as shown in Table 4, ‘free’ Pachysolen was unable to convert sugars to ethanol and no xylose was utilized. Immobilized Pachysolen converted most of the sugars (81%) to ethanol and converted 51% of the xylose. The data shows that immobilization greatly increased the ability of Pachysolen to overcome the inhibitory effects of the toxic compounds contained in the pH adjusted hydrolysate.

In overlimed hydrolysate “A”, as reflected in Table 4, ‘free’ Pachysolen converted 60% of sugars to ethanol, and immobilized Pachysolen 86% of sugars. Xylose utilization was 0% for free cells. This is a surprising result with respect to reports in the current literature that Pachysolen tannophilus will ferment pentoses, and particularly xylose, in a defined medium. It is the inventors' hypothesis that despite removal of detectable levels of HMF and furfural by overliming, significant amounts of other inhibitors, discussed above, or combinations thereof still remain in the hydrolysate thus preventing fermentation. When the Pachysolen tannophilus was immobilized xylose utilization jumped to 76%. Immobilization thus enhances the benefit of overliming and greatly increases xylose utilization.

Table 5 shows similar results to Table 4. In pH adjusted hydrolysate “B”, as shown in Table 5, ‘free’ Pachysolen was unable to convert sugars to ethanol and no xylose was utilized. Immobilized Pachysolen converted a majority of the sugars (57%) to ethanol.

Moreover, as reflected in Table 4, in overlimed hydrolysate “B” that contained very high inhibitor concentrations ‘free’ Pachysolen was unable to ferment available sugars, while immobilized Pachysolen fermented 83% of available sugars, including xylose, to ethanol.

TABLE 4
Softwood hydrolysate ‘A’ fermentation characteristics with Pachysolen tannophilus

| | pH adjusted | | | | | Overlimed and pH adjusted | | | | |
|---|---|---|---|---|---|---|---|---|---|---|
| | Ethanol yield | Sugar Utilization | | Solution | | Ethanol yield | Sugar Utilization | | Solution | |
| | (% | (%) | | Inhibitors (g/L) | | (% | (%) | | Inhibitors (g/L) | |
| Cells | theoretical)† | glucose | xylose | furfural | HMF | theoretical)† | glucose | xylose | furfural | HMF |
| Free | 0.0% | 0.0% | 0.0% | 0.42 | 4.03 | 61.8% | 72.2% | 0.0% | <DL | <DL |
| | | | | | | 57.4% | 75.8% | 0.0% | | |
| Imm. | 81.3% | 65.2% | 51.1% | 0.42 | 4.03 | 79.7% | 61.6% | 79.3% | <DL | <DL |
| | | | | | | 92.7% | 67.9% | 73.4% | | |

†Glucose concentration: 13.5 g/L;
Xylose concentration: 3.4 g/L
DL = Detectable Limit;
Imm. = Immobilized

TABLE 5
Softwood hydrolysate ‘B’ fermentation characteristics with Pachysolen tannophilus

| | pH adjusted | | | | | Overlimed and pH adjusted | | | | |
|---|---|---|---|---|---|---|---|---|---|---|
| | Ethanol yield | Sugar Utilization | | Solution | | Ethanol yield | Sugar Utilization | | Solution | |
| | (% | (%) | | Inhibitors (g/L) | | (% | (%) | | Inhibitors (g/L) | |
| Cells | theoretical)† | glucose | xylose | furfural | HMF | theoretical)† | glucose | xylose | furfural | HMF |
| Free | 0.0% | 0.0% | 0.0% | 5.91 | 1.32 | 0.0% | 0.0% | 0.0% | 1.04 | 0.79 |
| Imm | 56.9% | 35.7% | 15.9% | 5.91 | 1.32 | 83.3% | 53.8% | 73.0% | 1.04 | 0.79 |

†Glucose concentration: 4.7 g/L;
Xylose concentration: 3.2 g/L
Imm. = Immobilized

In the preceding example summarized in Table 4 and 5, ethanol yield (% theoretical) is based on glucose and xylose only and is calculated from total glucose and xylose concentrations before treatment. Other monosaccharides are not considered. All sugar utilization data is calculated using YSI results for glucose and xylose. Sugar utilization calculations do not differentiate between end products (i.e., includes ethanol, xylitol, biomass) and is calculated as follows (accounting for lost sugars after treatment like overliming, autoclaving, etc.):

### For Hydrolysate Calculations:

$$\% Sugar \times Conversion = \frac{NS - RS}{TS} * 95$$

$$N S = Sugar \times Concentration after Treatment ( i . e . , Negative Control )$$

$$R S = Residual Sugar \times Concentration after Fermentation$$

$$T S = Total Sugar \times Concentration before Treatment$$

In another example of the processes taught in the present patent document, the microbe/calcium alginate beads were re-used in sequential fermentations and the microbes in the beads were metabolically ‘regenerated’ between fermentations to increase ethanol yield.

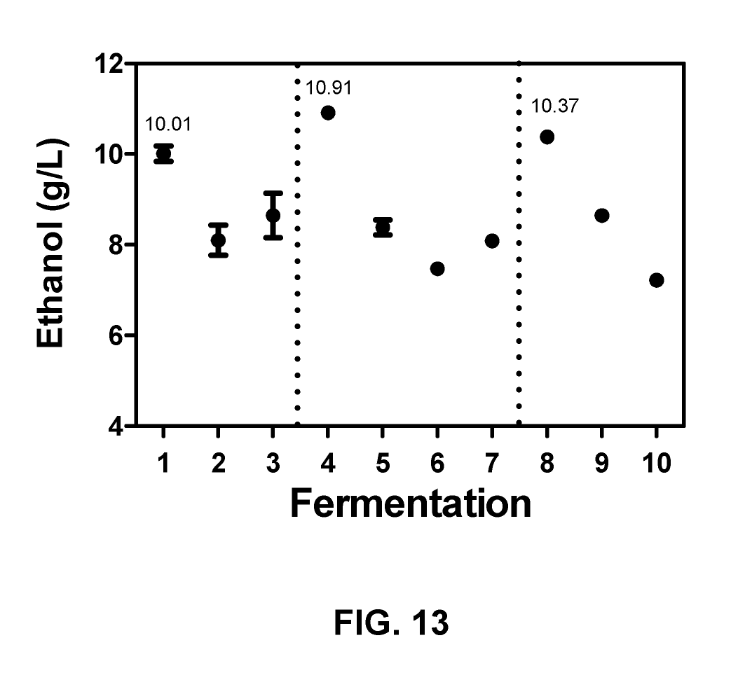

For the present example, fermentations using 2 g Pachysolen/calcium alginate beads per 10 ml softwood hydrolysate supplemented with 0.2% Urea, 0.2% Yeast Extract, and 0.05% KH₂PO₄ were performed at 30° C. and 75 rpm for 72 hours. After the fermentation reaction (Fermentation 1), the liquid was aseptically removed and analyzed for ethanol content, and the beads were aseptically rinsed several times with sterile deionized water. The same Pachysolen/calcium alginate beads were used in a second fermentation (Fermentation 2), in the same conditions, as Fermentation 1. Similarly, the fermentation liquid was subsequently analyzed and the beads rinsed. This was repeated for Fermentation 3. FIG. 13 illustrates the decreased ethanol yield in Fermentations 2 and 3 compared to Fermentation 1.

Next, the same Pachysolen/calcium alginate beads were regenerated between Fermentations 3 and 4 (shown as a dotted line in FIG. 13 between Fermentations 3 and 4) by incubating for 22 hours in a shaking incubator at 30° C. and 100 rpm in a yeast culture medium, Yeast Peptone Dextrose (YPD), after washing. The YPD was then aseptically removed and the beads were used in yet another fermentation (Fermentation 4). FIG. 13 illustrates that the regeneration of the Pachysolen/calcium alginate in culture medium restored the fermentative ability of the Pachysolen to produce ethanol.

Similar washes, fermentations, and a second regeneration (shown as a dotted line between fermentations 7 and 8) were performed using the same beads in another 6 fermentations. The results are shown in FIG. 13. FIG. 13 shows that immobilized microbes may be used in sequential fermentations and that the Pachysolen in the beads can be metabolically regenerated. Although the present example employs a regeneration step after 3 or 4 consecutive uses of the immobilized microbes, it is possible to regenerate the microbes more or less often. It is expected that if a greater number of beads are used in sequential fermentations (i.e. fermenting under conditions of a saturating yeast concentration), the ethanol yields would remain at a higher level in successive fermentations before requiring metabolic regeneration.

As discussed above, the immobilization medium, for example calcium alginate, can degrade due to use. If the microbes are regenerated and re-used according to the present example, it may be necessary to recycle the immobilization medium as taught above.

Although the invention has been described with reference to preferred embodiments and specific examples, it will readily be appreciated by those skilled in the art that many modifications and adaptations of the methods and bioreactors described herein are possible without departure from the spirit and scope of the invention as claimed hereinafter. Thus, it is to be clearly understood that this description is made only by way of example and not as a limitation on the scope of the invention as claimed below.

What is claimed is:

1. A process for converting wood into ethanol and paper pulp, the process comprising the steps of:
a. producing a liquid hydrolysate comprising hemicellulose hydrolysate and a biomass residue from debarked wood chips;
b. separating liquid hydrolysate from the biomass residue;
c. fermenting monosaccharides in the separated liquid hydrolysate using immobilized Pachysolen tannophilus; and
d. removing lignin from the biomass residue to form a paper pulp.

2. The process according to claim 1, wherein the producing step comprises the step of cooking the wood chips in a pressure reactor.

3. The process according to claim 1, wherein the separating step comprises pressing the biomass residue to express a portion of the liquid hydrolysate from the biomass residue.

4. The process according to claim 2, wherein the separating step comprises pressing the cooked wood chips to express a portion of the liquid hydrolysate from the cooked wood chips and the cooked wood chips comprise the biomass residue.

5. The process according to claim 1, wherein the Pachysolen tannophilus is immobilized in calcium alginate.

6. The process according to claim 5, wherein the calcium alginate is in the form of beads.

7. The process according to claim 6, wherein the calcium alginate beads have a diameter of about 3 mm.

8. The process according to claim 1, wherein more than 80% of the monosacchamides in the separated liquid hydrolysate are converted to ethanol.

9. A process for converting wood into ethanol and biofuel, the process comprising the steps of:
a. producing a liquid hydrolysate comprising hemicellulose hydrolysate and a biomass residue from debarked wood chips;
b. separating liquid hydrolysate from the biomass residue;
c. forming a solid high-energy density product from the biomass residue; and
d. fermenting monosaccharides in the separated hydrolysate.

10. The process according to claim 9, wherein the separating step comprises pressing the biomass residue to express a portion of the liquid hydrolysate from the biomass residue.

11. A high-energy density biofuel comprising a compacted biomass residue including cellulose and lignin and which is substantially free of hemicellulose, the compacted biomass residue having an energy density greater than 7,000 Btu/lb.

12. A high-energy density biofuel according to claim 11, wherein the compacted biomass has a water content of less than 25%.

13. A high-energy density biofuel according to claim 11, wherein the compacted biomass has a water content of less than about 45%

14. A high-energy density biofuel according to claim 13, wherein the compacted biomass comprises 10% or less by weight of hemicellulose.

15. A high-energy density biofuel according to claim 14, wherein the compacted biomass has an energy density greater than 7,500 Btu/lb.

16. A process for converting biomass fiber source into ethanol and a high value product, the process comprising the steps of:
a. producing a liquid hydrolysate comprising hemicellulose hydrolysate and a biomass residue from the biomass fiber source;
b. separating liquid hydrolysate from the biomass residue;
c. fermenting monosaccharides in the separated liquid hydrolysate using at least one microbial species immobilized in an immobilization medium; and
d. creating a high value product from the biomass residue.

17. The process of claim 16, wherein the biomass fiber source is bagasse and the high value product is a high-energy density product.

18. The process of claim 16, wherein the biomass fiber source is wood and the high value product is paper.

19. The process of claim 16, wherein the at least one microbial species includes a first microbial species and a second microbial species wherein a fermentation characteristic of the second microbial species is complimentary to a fermentation characteristic of the first microbial species.

20. The process of claim 19, wherein the first microbial species is a yeast and the second microbial species is a bacteria.

21. The process of claim 19, wherein the second microbial species more efficiently ferments a particular monosaccharide than the first microbial species.

22. The process of claim 19, wherein a metabolic rate with respect to a particular monosaccharide of the second microbial species is substantially different than the first microbial species.

23. The process of claim 16, wherein the at least one microbial species includes a first microbial species immobilized in a first immobilization medium and a second microbial species immobilized in a separate immobilization medium from the first.

24. The process of claim 23, wherein the first microbial species is contained in a first fermentation vessel and the second microbial species is contained in a second fermentation vessel wherein during the fermentation step the liquid hydrolysate is passed through both the first fermentation vessel and the second fermentation vessel.

25. The process according to claim 16, wherein the at least one microbial species includes a first microbial species and a second microbial species wherein both the first microbial species and the second microbial species are immobilized together in a calcium alginate bead.

26. The process according to claim 16, wherein after the separating step the process further comprising the step of:
conditioning the liquid hydrolysate.

27. The process according to claim 26, wherein the conditioning step includes removing a high value product from the biomass hydrolysate.

28. The process according to claim 27, wherein the high value product is selected from a group consisting of sulfuric acid, acetic acid, phenolic compounds, polyphenolic compounds, nutraceutical products, cosmeceutical products, pharmaceutical products, furans, furfural, and 5-hydroxymethylfurfural.

29. The process according to claim 27, wherein the high value product is removed by adsorption.

30. The process according to claim 27, wherein the high value product is removed by filtration.

31. A process for converting sugarcane bagasse into ethanol and a high-energy density biofuel, the process comprising the steps of:
a. producing a liquid hydrolysate comprising hemicellulose hydrolysate and a biomass residue from sugarcane bagasse;
b. separating liquid hydrolysate from the biomass residue;
c. fermenting monosaccharides in the separated liquid hydrolysate using immobilized microbes; and
d. reducing the moisture content of the biomass residue to produce a high-energy density biofuel.

32. The process according to claim 31, wherein after the separating step the process further comprising the step of:
recovering fermentation inhibiting high value products from the liquid hydrolysate.
